(19) United States
(12) Patent Application Publication
(10) Pub. No.: US 2023/0248715 A1
(43) Pub. Date: Aug. 10, 2023

(54) METHOD OF TREATING ACUTE MIGRAINE WITH CGRP-ACTIVE COMPOUND

(71) Applicant: Merck Sharp & Dohme Corp., Rahway, NJ (US)

(72) Inventors: Chi-Chung Liu, Rahway, NJ (US); David Michelson, North Wales, PA (US); Gene Marcantonio, Kenilworth, NJ (US); Kyle Fliszer, North Wales, PA (US); Rebecca White, West Point, PA (US); Mark E. Fraley, West Point, PA (US); Leonardo R. Allain, West Point, PA (US); Tiffani Voss, North Wales, PA (US); John Limanto, Rahway, NJ (US)

(21) Appl. No.: 18/137,949

(22) Filed: Apr. 21, 2023

Related U.S. Application Data
(63) Continuation of application No. 15/719,771, filed on Sep. 29, 2017.
(60) Provisional application No. 62/402,560, filed on Sep. 30, 2016.

(51) Int. Cl. A61K 31/4545; A61K 9/16; A61P 25/06; A61K 9/20

(52) U.S. Cl. CPC A61K 31/4545; A61K 9/1652; A61P 25/06; A61K 9/2054

(57) ABSTRACT

A method of alleviating or mitigating at least one symptom of migraine attack by administering to a patient suffering from a migraine attack a therapeutically effective amount of the compound of Formula I:

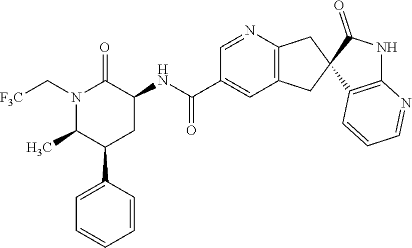 [SMILES: C[C@@H]1[C@H](c2ccccc2)C[C@H](NC(=O)c2cnc3c(c2)C[C@@]2(C3)C(=O)Nc3ncccc32)C(=O)N1CC(F)(F)F]

or a pharmaceutically acceptable salt thereof.

### CROSS-REFERENCE TO RELATED APPLICATIONS

This application is a continuation of U.S. patent application Ser. No. 15/719,771, filed Sep. 29, 2017; which claims the benefit of U.S. Provisional Patent Application No. 62/402,560, filed Sep. 30, 2016, the entire contents of each of which are herein incorporated by reference in their entirety.

### BACKGROUND OF THE INVENTION

Migraine represents a significant burden to patients and society. Triptans have been widely used in migraine treatment but a substantial number of patients do not respond to triptan therapy. Lack of efficacy can lead to conversion of episodic migraine to chronic migraine (see for example Serrano et al., Effects of Switching Acute Treatment on Disability in Migraine Patients Using Triptans, Headache 2013; 53: 1415-1429 and Lipton et al., Impact of NSAID and Triptan Use on Developing Chronic Migraine, Headache, 2013: 53: 1548-1563). Triptans are also agonists of 5-HT receptors which implicate them as potential arterial constrictors, contraindicating their use by patients having comorbid cardiovascular conditions. Such co-morbidity increases in patient populations of increasing age. Accordingly, therapeutics useful in the management of migraine in aging populations become increasingly difficult to identify

What is needed are therapies for managing migraine conditions which are based on new modes of action and are without cardiovascular risks and contraindications.

CGRP (Calcitonin Gene-Related Peptide) is a naturally occurring 37-amino acid peptide that is generated by tissue-specific alternate processing of calcitonin messenger RNA and is widely distributed in the central and peripheral nervous system. Calcitonin gene-related peptide (CGRP) is a potent vasodilatory neurotransmitter believed to play a key role in migraine pathophysiology. The initial human clinical validation of the CGRP target was provided by Boehringer Ingelheim in 2003 with the report that an IV formulation comprising olcegepant was efficacious in the acute treatment of migraine and the mechanism was confirmed by a study using telcagepant (a CGRP antagonist) in an oral formulation.

### SUMMARY OF THE INVENTION

Newly developed compounds demonstrating CGRP receptor antagonist properties have been described, for example, compounds described in published international application, publication no. WO 2012/064910, for example, the compound having the structure of Formula I (ubrogepant):

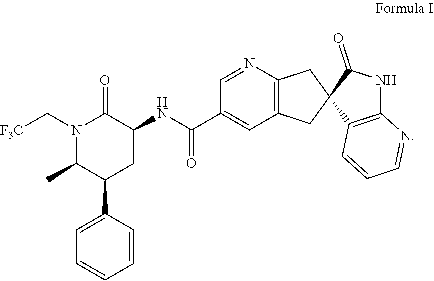 [SMILES: C[C@@H]1[C@H](c2ccccc2)C[C@H](NC(=O)c2cnc3c(c2)C[C@@]2(C3)C(=O)Nc3ncccc32)C(=O)N1CC(F)(F)F]

Now it has been surprisingly found that administration of ubrogepant is well-tolerated, and a potent CGRP-antagonist with low potential for side effects and metabolic complications.

Accordingly, in one aspect the present invention is a method of alleviating one or more symptoms (at least one symptom) of acute migraine attack, the method comprising administration of ubrogepant, or a pharmaceutically acceptable salt or prodrug thereof, preferably ubrogepant or a pharmaceutically acceptable salt thereof, in an amount sufficient to alleviate at least one symptom of an acute migraine attack. In some embodiments it is preferred to administer an amount of ubrogepant or a pharmaceutically acceptable salt thereof, which alleviates at least one symptom of acute migraine attack within two hours. In some embodiments it is preferred to administer at least 25 mg of ubrogepant, or a salt or prodrug thereof having a potency equivalent to 25 mg of ubrogepant, preferably ubrogepant or a pharmaceutically acceptable salt thereof. In some embodiments it is preferred to administer at least 50 mg of ubrogepant, or a salt or prodrug thereof having a potency equivalent to 50 mg of ubrogepant, preferably ubrogepant or a pharmaceutically acceptable salt thereof. In some embodiments it is preferred to administer at least 100 mg of ubrogepant, or a salt or prodrug thereof having a potency equivalent to 100 mg of ubrogepant, preferably ubrogepant or a pharmaceutically acceptable salt thereof.

In some embodiments of the invention the symptom alleviated is at least one of: (i) pain (ii) aura; (iii) photophobia; (iv) phonophobia; (v) nausea; or (vi) emesis.

In one aspect the present invention is a method of alleviating the pain symptom of an acute migraine attack within two hours by administering to a patient experiencing pain from an acute migraine attack a formulation comprising at least 50 mg of ubrogepant, or a pharmaceutically acceptable salt or prodrug thereof having a potency equivalent to 50 mg of ubrogepant, preferably ubrogepant or a pharmaceutically acceptable salt thereof. In some embodiments it is preferred to administer a formulation comprising an amount of ubrogepant, or a pharmaceutically acceptable salt or prodrug thereof having a potency equivalent to 100 mg of ubrogepant, preferably ubrogepant or a pharmaceutically acceptable salt thereof.

### BRIEF DESCRIPTION OF THE DRAWINGS

FIG. 1: Flow Chart Illustrating Study Design

FIG. 2: Chart Illustrating Baseline Symptoms Experienced by Participants in Study at Onset of Migraine Symptoms

FIG. 3: Chart Illustrating Results In Participants In Study Post-Dosing Broken Down By Symptom and Primary Endpoint Results

FIG. 4A: Chart Illustrating Proportion of Participants Reporting Pain Freedom at Two Hours

Post-dose by Treatment Group

FIG. 4B Chart Illustrating Proportion of Participants Reporting Headache Response at Two Hours Post-dose by Treatment Group

### DETAILED DESCRIPTION OF THE INVENTION

Ubrogepant is believed to have utility in treating patients suffering from acute migraine attack, specifically, ubrogepant is believed to be useful in alleviating (that is, diminishing in severity or eliminating the experience of) one or more symptoms associated with acute migraine attack. Patients suffering from acute migraine, as the term is used herein, typically present with one or more of the following symptoms: (i) pain; (ii) aura; (iii) photophobia; (iv) phonophobia; (v) nausea; and (vi) emesis. In general, not every patient suffering migraine attack may experience each of these symptoms during an attack, however, in general the pain symptom is present to some degree in all patients suffering from an acute migraine attack.

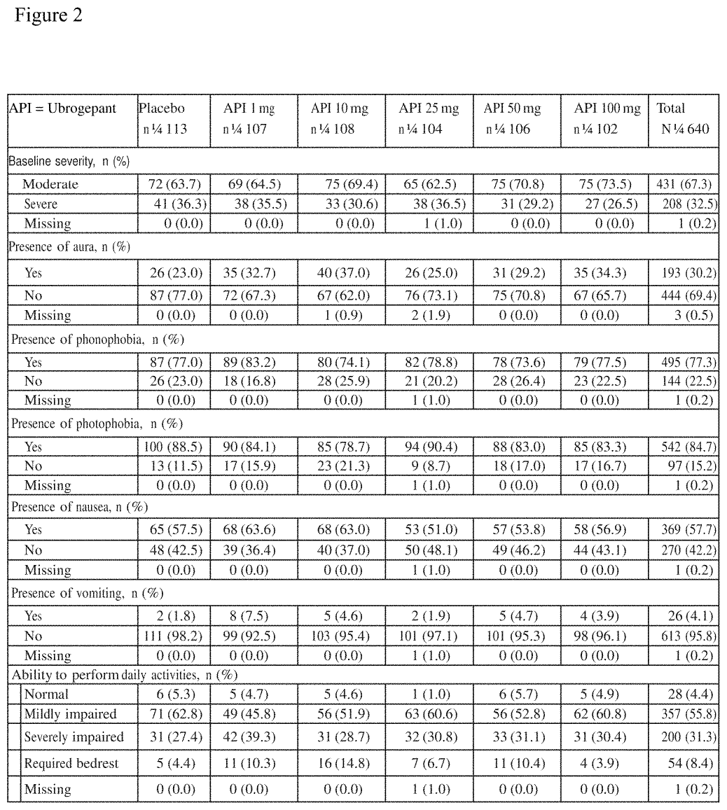

While all of the symptoms listed above may not be present in a patient suffering from a particular incidence of acute migraine attack, in general each of the symptoms presented in a specific patient during a particular migraine attack may be experienced by the patient at varying degrees of affliction. In general, patients are asked to keep a diary recording the presence or absence of a particular symptom in a set of symptoms associated with migraine attack (see for Example FIG. 2). Clinicians typically instruct a patient to rate the level of severity of a particular symptom on a scale of 4 categories. In general the categories range from a particular symptom being “not present”, through the lowest level at which a symptom is being experienced (e.g. a category of “mildly impaired”), an intermediate level (severely impaired), and the highest level of the symptom experience incapacitating the patients normal functionality (requiring bedrest). Clinicians may ascribe an arbitrary number system associated with these 4 levels of symptom experience, the lowest numerical rating equivalent to “not present” and the highest level equivalent to “incapacitating” levels of a symptom being experienced by a patient suffering an acute migraine attack. In general the endpoint of the study considers whether, within the specified time window from

As used herein, unless otherwise specified, the following terms have the following meanings:

Unless expressly stated to the contrary, all ranges cited herein are inclusive; i.e., the range includes the values for the upper and lower limits of the range as well as all values in between. As an example, temperature ranges, percentages, ranges of equivalents, and the like described herein include the upper and lower limits of the range and any value in the continuum there between.

The phrase “at least one” used in reference to the number of components comprising a formulation, for example, “at least one pharmaceutical excipient” means that one member of the specified group is present in the formulation, and more than one may additionally be present. Components of a formulation are typically aliquots of isolated pure material added to the formulation, where the purity level of the isolated material added into the formulation is the normally accepted purity level for a reagent of the type. The phrase “one or more”, means the same as “at least one”;

As used herein, the term “formulation” is intended to encompass a product comprising ubrogepant and other specified ingredients in the specified amounts, as well as any product which results, directly or indirectly, from combination of the specified ingredients in the specified amounts.

“concurrently” and “contemporaneously” both include in their meaning (1) simultaneously in time (e.g., at the same time); and (2) at different times but within the course of a common treatment schedule;

“consecutively” means one following the other;

“sequentially” refers to a series administration of therapeutic agents that awaits a period of efficacy to transpire between administering each additional agent; this is to say that after administration of one component, the next component is administered after an effective time period after the first component; the effective time period is the amount of time given for realization of a benefit from the administration of the first component;

“effective amount” or “therapeutically effective amount” is meant to describe the provision of an amount of ubrogepant which is effective in alleviating at least one symptom, as discussed herein, of an acute migraine attack. For example, in treating acute migraine pain with ubrogepant, an “effective amount” of ubrogepant (or “therapeutically effective amount” of ubrogepant) means, for example, providing that amount of ubrogepant that results in a therapeutic response in a patient suffering from an acute migraine attack. In some embodiments the symptom is pain and the degree of alleviation is the reduction of pain to a level permitting the patient to engage in normal activities. In some embodiments an effective amount of ubrogepant is that amount which reduces the severity of symptoms present in a patient suffering an acute migraine attack within two hours of administration.

“patient” is a subject afflicted with a condition to be treated, and “subject” means an animal, such as a mammal (e.g., a human being) and is preferably a human being;

As the term in all of its forms is used herein, “alleviate”, “alleviation”, and “alleviated” means that a particular symptom present in an acute migraine attack is lessened in severity. In some embodiments a symptom is alleviated to a level of affliction that allows the treated patient to return to participating in normal activity (lowest level at which a symptom is present), or to be free of that symptom altogether. In some embodiments, treatment is considered successful if a symptom is alleviated within two hours post administration of a dose of ubrogepant. In some embodiments treatment is considered successful if pain is alleviated to the lowest level at which a symptom is present or is eliminated altogether within two hours post ubrogepant administration with or without a concomitant reduction in severity of one or more other symptoms within the two hour post-administration window.

As described herein, unless otherwise indicated, the use of ubrogepant in treatment means that an amount of ubrogepant, generally presented as a component of a formulation that comprises other excipients, is administered in aliquots of an amount, and at time intervals, which provides and maintains at least a therapeutic serum level of at least one pharmaceutically active form of the compound over the time interval between dose administration.

Ubrogepant may be used alone in treatment and may also be used in combination with other pharmaceutically-active agents in treating a patient suffering from an acute migraine attack. Accordingly, ubrogepant may be administered in conjunction with another agent, for example, an ergotamine and dihydroergotamine, or other serotonin agonists, especially a 5-HT_{1B/1D} agonist, for example sumatriptan, naratriptan, zolmitriptan, eletriptan, almotriptan, frovatriptan, donitriptan, avitriptan, and rizatriptan, a 5-HT_{1D} agonist such as PNU-142633 and a 5-HT_{1F} agonist such as lasmiditan; a cyclooxygenase inhibitor, such as a selective cyclooxygenase-2 inhibitor, for example rofecoxib, etoricoxib, celecoxib, valdecoxib or paracoxib; a non-steroidal anti-inflammatory agent or a cytokine-suppressing anti-inflammatory agent, for example with a compound such as ibuprofen, ketoprofen, fenoprofen, naproxen, indomethacin, sulindac, meloxicam, piroxicam, tenoxicam, lornoxicam, ketorolac, etodolac, mefenamic acid, meclofenamic acid, flufenamic acid, tolfenamic acid, diclofenac, oxaprozin, apazone, nimesulide, nabumetone, tenidap, etanercept, tolmetin, phenylbutazone, oxyphenbutazone, diflunisal, salsalate, olsalazine or sulfasalazine and the like; or glucocorticoids. Similarly, the instant compounds may be administered with an analgesic such as aspirin, acetaminophen, phenacetin, fentanyl, sufentanil, methadone, acetyl methadol, buprenorphine or morphine.

Additionally, ubrogepant may be used in conjunction with an interleukin inhibitor, such as an interleukin-1 inhibitor; an NK-1 receptor antagonist, for example aprepitant; an NMDA antagonist; an NR2B antagonist; a bradykinin-1 receptor antagonist; an adenosine A1 receptor agonist; a sodium channel blocker, for example lamotrigine; an opiate agonist such as levomethadyl acetate or methadyl acetate; a lipoxygenase inhibitor, such as an inhibitor of 5-lipoxygenase; an alpha receptor antagonist, for example indoramin; an alpha receptor agonist; a vanilloid receptor antagonist; a renin inhibitor; a granzyme B inhibitor; a substance P antagonist; an endothelin antagonist; a norepinephrin precursor; anti-anxiety agents such as diazepam, alprazolam, chlordiazepoxide and chlorazepate; serotonin 5HT₂ receptor antagonists; opioid agonists such as codeine, hydrocodone, tramadol, dextropropoxyphene and febtanyl; an mGluR5 agonist, antagonist or potentiator; a GABA A receptor modulator, for example acamprosate calcium; nicotinic antagonists or agonists including nicotine; muscarinic agonists or antagonists; a selective serotonin reuptake inhibitor, for example fluoxetine, paroxetine, sertraline, duloxetine, escitalopram, or citalopram; an antidepressant, for example amitriptyline, nortriptyline, clomipramine, imipramine, venlafaxine, doxepin, protriptyline, desipramine, trimipramine, or imipramine; a leukotriene antagonist, for example montelukast or zafirlukast; an inhibitor of nitric oxide or an inhibitor of the synthesis of nitric oxide.

Ubrogepant may be used also in conjunction with gap junction inhibitors; neuronal calcium channel blockers such as civamide; AMPA/KA antagonists such as LY293558; sigma receptor agonists; and vitamin B2. Additionally, ubrogepant may be used in conjunction with ergot alkaloids other than ergotamine and dihydroergotamine, for example, ergonovine, methylergonovine, metergoline, ergoloid mesylates, dihydroergocornine, dihydroergocristine, dihydroergocryptine, dihydro-α-ergocryptine, dihydro-β-ergocryptine, ergotoxine, ergocornine, ergocristine, ergocryptine, α-ergocryptine, β-ergocryptine, ergosine, ergostane, bromocriptine, or methysergide.

Additionally, ubrogepant may be used in conjunction with a beta-adrenergic antagonist such as timolol, propanolol, atenolol, metoprolol or nadolol, and the like; a MAO inhibitor, for example phenelzine; a calcium channel blocker, for example flunarizine, diltiazem, amlodipine, felodipine, nisolipine, isradipine, nimodipine, lomerizine, verapamil, nifedipine, or prochlorperazine; neuroleptics such as olanzapine, droperidol, prochlorperazine, chlorpromazine and quetiapine; an anticonvulsant such as topiramate, zonisamide, tonabersat, carabersat, levetiracetam, lamotrigine, tiagabine, gabapentin, pregabalin or divalproex sodium; an anti-hypertensive such as an angiotensin II antagonist, for example losartan, irbesartin, valsartan, eprosartan, telmisartan, olmesartan, medoxomil, candesartan and candesartan cilexetil, an angiotensin I antagonist, an angiotensin converting enzyme inhibitor such as lisinopril, enalapril, captopril, benazepril, quinapril, perindopril, ramipril and trandolapril; or botulinum toxin type A or B.

In addition, ubrogepant may be used in conjunction with a potentiator such as caffeine, an H2-antagonist, simethicone, aluminum or magnesium hydroxide; a decongestant such as oxymetazoline, epinephrine, naphazoline, xylometazoline, propylhexedrine, or levo-desoxy-ephedrine; an antitussive such as caramiphen, carbetapentane, or dextromethorphan; a diuretic; a prokinetic agent such as metoclopramide or domperidone; a sedating or non-sedating antihistamine such as acrivastine, azatadine, bromodiphenhydramine, brompheniramine, carbinoxamine, chlorpheniramine, clemastine, dexbrompheniramine, dexchlorpheniramine, diphenhydramine, doxylamine, loratadine, phenindamine, pheniramine, phenyltoloxamine, promethazine, pyrilamine, terfenadine, triprolidine, phenylephrine, phenylpropanolamine, or pseudoephedrine. The present compounds also may be used in conjunction with anti-emetics.

The above combinations include combinations of ubrogepant with two or more other active compounds, including in combination with other drugs that are used in the treatment of acute migraine attack. Such other drug(s) may be administered, by a route and in an amount commonly used therefor, contemporaneously or sequentially with ubrogepant. When ubrogepant is used contemporaneously with one or more other drugs, a pharmaceutical formulation in unit dosage form containing such other drugs and ubrogepant is preferred. However, the combination therapy may also include therapies in which ubrogepant and one or more other drugs are administered on different overlapping schedules.

The present invention includes within its scope prodrugs of ubrogepant. In general, such prodrugs will be functional derivatives of ubrogepant which are readily converted in vivo into ubrogepant or an active metabolite thereof. Thus, in the methods of treatment of the present invention, the terms “administration of” or “administering” ubrogepant shall encompass the treatment of the various conditions described with the compound specifically disclosed or with a compound which may not be specifically disclosed, but which converts to ubrogepant or an active metabolite in vivo after administration to the patient. Conventional procedures for the selection and preparation of suitable prodrug derivatives are described, for example, in “Design of Prodrugs,” ed. H. Bundgaard, Elsevier, 1985. Metabolites of these compounds include active species produced upon introduction of compounds of this invention into the biological milieu.

The term “formulation”, as used herein, refers to a blend, aggregation, solution or other combination of materials which includes ubrogepant, or a pharmaceutically acceptable salt thereof, or a form of ubrogepant which is converted in vivo into ubrogepant or a pharmaceutically acceptable salt or pharmaceutically active metabolite thereof (also described herein as an active pharmaceutical ingredient, API), which formulation is adapted to a particular mode of administration (suitable to be incorporated into a dosage form facilitating that mode of administration). For example, a formulation suitable for pressing into tablets designed for oral administration, in the treatment of a patient suffering an acute migraine attack. It will be appreciated that alternative formulation which facilitate other modes of administering ubrogepant in an amount providing an equivalent to the therapeutic serum level of ubrogepant provided by the formulation described in detail herein may alternatively be employed. It will be appreciated also that reference herein to a particular weight of ubrogepant (the compound of Formula I) in a particular formulation is equivalent to an amount of a different form of ubrogepant (e.g., a salt thereof or a prodrug thereof) which represents the same potency (provides the same serum level of ubrogepant or an equivalently therapeutically active metabolite thereof) when released in vivo in a subject.

“Formulation”, as used herein is intended to encompass a product comprising ubrogepant and the other specified excipients, in the specified amounts, as well as any product which results, directly or indirectly, from combination of the specified excipients with ubrogepant in the specified amounts. Such term in relation to pharmaceutical formulation, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical formulations of the present invention encompass any composition made by admixing ubrogepant and a pharmaceutically acceptable carrier. By “pharmaceutically acceptable” it is meant the carrier, diluent or excipient must be compatible with the other ingredients of the formulation and not deleterious to the recipient thereof.

As will be appreciated, pharmaceutical formulations can be solid, semi-solid or liquid. Solid form preparations can be adapted to a variety of modes of administration, examples of which include, but are not limited to, powders, dispersible granules, mini-tablets, beads, which can be used, for example, for tableting, encapsulation, or direct administration. Liquid form preparations include, but are not limited to, solutions, suspensions and emulsions which for example, but not exclusively, can be employed in the preparation of formulations intended for parenteral injection, for intranasal administration, or for administration to some other mucosal membrane. Formulations prepared for administration to various mucosal membranes may also include additional components adapting them for such administration, for example, viscosity modifiers.

For preparing pharmaceutical formulations containing a pharmaceutically active form of ubrogepant (e.g., the free-base or a pharmaceutically acceptable salt or prodrug thereof) will be combined with one or more pharmaceutically acceptable excipients. These excipients impart to the formulation properties which make it easier to handle or process, for example, lubricants or pressing aids in powdered medicaments intended to be tableted, or adapt the formulation to a desired route of administration. Various routes of administration include, for example, excipients which provide a formulation for oral administration, for example, via absorption from the gastrointestinal tract, those suitable for transdermal or transmucosal administration, for example, via adhesive skin “patch”, those suitable for buccal administration, or those suitable for injection, for example, intramuscular or intravenous, routes of administration. These excipients are collectively termed herein “a carrier”. In general, the pharmaceutical formulations are prepared by uniformly and intimately bringing ubrogepant into association with a carrier, either a liquid carrier or a finely divided solid carrier or both. Thus, pharmaceutical formulations containing ubrogepant in any therapeutically active form may be in any form suitable for the intended route of administration, including, for example, the tablets described herein or variations thereof, troches, lozenges, aqueous or oily suspensions, dispersible powders or granules, emulsions, solutions, hard or soft capsules, or syrups or elixirs.

Examples of pharmaceutically acceptable carriers and methods of manufacture for various formulations mentioned herein may be found in A. Gennaro (ed.), Remington: The Science and Practice of Pharmacy, 20th Edition, (2000), Lippincott Williams & Wilkins, Baltimore, Md.

The weight ratios of ubrogepant, other excipients, and any other active compounds in a pharmaceutical formulation suitable for use in the present invention may be varied and will depend upon the effective dose of each ingredient. In addition, where ubrogepant is administered with other active compounds, the active compounds administered may equivalently be administered separately, either contemporaneously or simultaneously, as well as be present in a single dosage form with the other active compounds (combination dosage form). It will be appreciated that contemporaneous or simultaneous administration is equally well accomplished by administering multiple active compounds via the same or by different administration routes, so long as the administration route provides a therapeutic serum level intended for the active compounds administered.

Alternate formulations may include aqueous suspensions containing ubrogepant and any other active compounds in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending ubrogepant in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These formulations may be preserved by the addition of an anti-oxidant such as ascorbic acid.

Dispersible powders and granules suitable for preparation of an aqueous suspension by the addition of water at the point of administration may be provided in the form of ubrogepant in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical formulations of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents.

While formulations of the invention may be employed in bulk form, it will be appreciated that for most applications the inventive formulations will be incorporated into a dosage form suitable for administration to a patient, each dosage form comprising an amount of the selected formulation which provides upon administration to a patient an effective amount of ubrogepant or an active metabolite thereof. Examples of suitable dosage forms include, but are not limited to, dosage forms adapted for: (i) oral administration, e.g., a liquid, gel, powder, solid or semi-solid pharmaceutical formulation which is loaded into a capsule or pressed into a tablet and may comprise additionally one or more coatings which modify its release properties, for example, coatings which impart delayed release or formulations which have extended release properties.

As will be appreciated, medication for treatment of migraine symptoms is desirably in the form of an oral medication providing an acceptable amount of the API (ubrogepant) to alleviate (as defined herein) one or more symptoms of migraine attack. Due to the nature of migraine attacks, including its rapid onset and debilitating symptoms, it is desirable to provide a therapeutic amount of ubrogepant to a patient to whom it is being administered as rapidly as possible.

Pharmaceutical formulations intended for oral use may be prepared according to any method known to the art for the manufacture of pharmaceutical formulations and such formulations may contain ubrogepant (or an equivalently pharmaceutically active salt or prodrug thereof), optionally other active compounds, as described herein, and one or more excipients described above and additional ingredients, for example, those selected from the group consisting of sweetening agents, flavoring agents, coloring agents and preserving agents in order to provide pharmaceutically elegant and palatable preparations. The pharmaceutical formulations for the administration of ubrogepant may conveniently be presented in dosage unit form and may be prepared by any of the methods well known in the art of pharmacy. Formulations and dosage forms may include in addition to ubrogepant, and optionally one or more other active ingredients such as are described above, in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia; and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release. Oral tablets may also be formulated for immediate release, such as fast melt tablets or wafers, rapid dissolve tablets or fast dissolve films.

Formulations for oral use may also be presented as hard gelatin capsules wherein the active ingredient is mixed with an inert solid diluent, for example, calcium carbonate, calcium phosphate or kaolin, and charged directly into the capsule, or prepared as soft gelatin capsules wherein the active ingredient is mixed with water or an oil medium, for example peanut oil, liquid paraffin, or olive oil.

Since ubrogepant is directed to treatment of acute migraine, a rapid-release formulation is believed to be important in providing a therapeutic benefit to human patients to whom such a formulation is administered. In recent clinical studies a spray-dried formulation containing ubrogepant was employed in the treatment of migraine attack in the form of an orally-administered tablet.

### Spray-Dried Granulate Formulation and Tablets Prepared Thereform

A matrix containing ubrogepant was prepared by spray-drying a solution of ubrogepant and hypromellose acetate succinate type LG ® (Shin Etsu, article of commerce), blending the dried granulate with various excipients and pressing the mixture into tablets.

Thus, into 14.96 kg of USPNF grade acetone, over 1 hour, was mixed 590.4 g of ubrogepant followed by the addition of 1254.6 g hypromellose acetate succinate-LG over a period of 2.5 hours. Stirring was continued for 1 hour following the last aliquot of solids addition. This solution was charged into a Bend Research Niro® PSD-1 spray-drying apparatus.

Granulate was prepared by operating the spray-drying apparatus supplied with 99.998% bulk nitrogen (article of commerce) under the following conditions: (i) the gas supply rate of from 1860 g/min to 1992 g/min over the 45 minutes the spray dryer was operated at temperature: (ii) dryer inlet temperature was maintained 105° C., ±3° C.; (iii) dryer outlet temperature was observed at 48° C., ±8° C. over the course of the run; (iv) solution feed rate maintained at 180 g/min ±10 g/min over the course of the run. This run provided 1697.2 g of spray-dried granulate.

The spray-dried granulate prepared in the previous step was loaded into a Gruenberg tray dryer at room temperature. The dryer was operated by ramping up to 40° C. over 30 minutes, holding the temperature at 40° C., ±5° C. for 6 hours, with a concomitant rise in observed humidity from 15% relative humidity to 45% relative humidity, then ramping the temperature down to 21° C. over a period of 45 min and holding for a period of 58 hours and 20 min., resulting in the preparation of 1.610 Kg of dried granulate.

The granulate prepared above was incorporated into a tablet containing 1 mg, 5 mg, 10 mg, or 50 mg equivalent of ubrogepant for oral administration using one of the following procedures.

For tablets having 1mg of ubrogepant potency the formulation was prepared by placing into a 3L twin shell Dry Blender 3.5 g of Silicon Dioxide that was co-sieved through a 30 mesh screen with 161.3 grams of Avicel PH102®. The charge was blended at 30 rpm for one minute. Into a mortar was placed 46.663 grams of Fast Flo Lactose 316 and 23.33 grams of the spray-dried granulate prepared above and the charge was triturated with a pestle for 2 minutes. Into the triturated mixture was placed an additional 69.990 g of Lactose 316 Fast Flo and the mixture was titurated for an additional 2 minutes, followed by a third additional of 140.0 grams of Lactose 316 Fast Flow® with two minutes of trituration, followed by a fourth addition of 65.954 g of Lactose 316 Fast Flow® and another 2 minute trituration period. Thus prepared, 345.9 g of this triturated preblend was added to the blender, the mortar was rinsed with 161.3 grams of Avicel® which was added to the blender, followed by 21.0 g of crosscarmelose sodium and the blender was run for 15 minutes at 30 rpm. At the end of 15 minutes of blending, 1.750 g of magnesium stearate (pre-screened with 60 mesh) was added to the blender and the blender was run for an additional 5 minutes yielding 689.3 grams of the mixture. This mixture was roller compacted using a Alexanderwerk WP 120, yielding 652.7 grams of roller-compacted granulate. The roller compacted granules were combined with 1.644 g of magnesium stearate (pre-screened with 60 mesh), and 3.288 g of colloidal silica (Cab-o-sil®), and this mixture was charged into the blender and blended for 5 minutes at 30 RPM. This mixture was pressed into tablets by placing 100 mg of the mixture into an 8/32″ standard concave round tablet die using a standard tablet press.

Preparation of a blend suitable for the pressing tablets having 5 mg. ubrogepant potency was carried out by co-sieving 10.0 g of Silicon Dioxide with 460.1 grams of Avicel® PH102 through a 30 mesh screen and charging the mixture into a blender and blending it at 20 rpm for 1 minute.

Into a mortar was placed 138.8 grams of Fast Flo Lactose 316 and 69.4 grams of the spray-dried granulate prepared above, and the charge was triturated with a pestle for 2 minutes. Into the triturated mixture was placed an additional 208.2 g of Lactose 316 Fast Flo and the mixture was titurated for an additional 2 minutes, followed by a third addition of 416.4 grams of Lactose 316 Fast Flow® with two minutes of trituration, followed by a fourth addition of 156.8 g of Lactose 316 Fast Flow® and another 2 minute trituration period. Thus prepared, 989.6 g of this triturated preblend was added to the blender, the mortar was rinsed with 460.1 grams of Avicel® which was added to the blender, followed by 60.0 g of crosscarmelose sodium and the blender was run for 20 minutes at 20 rpm. At the end of 20 minutes of blending, 5.001 g of magnesium stearate (pre-screened with 60 mesh) was added to the blender and the blender was run for an additional 5 minutes yielding 1977.0 grams of the mixture. This mixture was roller compacted using a Alexanderwerk WP 120, yielding 1873.5 grams of roller-compacted granulate. The roller compacted granules were combined with 4.719 g of magnesium stearate (pre-screened with 60 mesh), and 9.438 g of Cab-o-sil, the mixture was charged into the blender and blended for 5 minutes at 20 RPM. This mixture was pressed into tables by placing 100 mg of the mixture into an 8/32″ standard concave round tablet die using a standard tablet press.

Preparation of a blend suitable for the pressing tablets having either 10 mg or 50 mg. ubrogepant potency was carried out by co-sieving 25.0 g of Silicon Dioxide with 760.0 grams of Avicel PH102® through a 30 mesh screen and charging the mixture into a blender and blending it at 20 rpm for 1 minute. At the end of 1 minute, into the blender was charged an additional 760.0 grams of Avicel PH102®, 1735 g of the ubrogepant granulate prepared above, 150 g of crosscarmellose sodium (USP grade, article of commerce) and 1502 g of of Lactose 316 Fast Flow®, and the blender was operated at 20 rpm for an additional 20 minutes. magnesium stearate which had been sieved through a 60 mesh screen (12.5 g) was added into the blender and the blender was operated at 20 rpm for 5 minutes yielding 4948 g of blended material. This blended material was roller compacted using Alexanderwerk WP 120 roller compactor, yielding 4861.7 g of roller compacted material. This material was added into the blender along with 12.24 g of magnesium stearate which had been sieved through a 60 mesh screen along with 24.49 g of silicon dioxide which had been sieved through a 30 mesh screen, and the blender was operated at 20 RPM for 5 minutes, yielding 4895.1 g of tableting blend.

To prepare a tablet of 10 mg ubrogepant potency, a 100 mg aliquot of the blend was pressed in a standard tablet press using an 8/32″ standard concave round tablet die. To prepare a tablet of 50 mg ubrogepant potency, 500 mg aliquot of the blend was pressed in a standard tablet press using an 14/32″ standard concave round tablet die.

### Clinical Trials Conducted Using Spray-Dried Study Medication Prepared As Described Herein:

A Phase IIb, multicenter, randomized, double-blind, placebo-controlled trial was conducted (Merck Protocol MK-1602-006;published online at ClinicalTrials.gov as NCT01613248) to investigate the efficacy and safety of ubrogepant across a range of doses. The study was conducted in accordance with principles of Good Clinical Practice, and was approved by Schulman Associates Institutional Review Board Inc. Each participant provided written informed consent. This study complied with the International Headache Society's guidelines for controlled trials of drugs in migraine, including the use of two-hour pain freedom as an endpoint. The sample size was large, populations were well balanced, and demographics were reflective of the broader migraine populations, contributing to the generalizability of the trial.

Participants were allocated in a double-blind fashion using a computer-generated randomized allocation schedule prepared by a blinded statistician. Randomization was stratified based on the participant's self-reported usual triptan response: oral triptan high-responder (current users of triptans who respond 75% of the time); oral triptan low-responder (current users of triptans who respond <75% of the time or those who have tried triptans but no longer use them); oral triptan naive. Numbered containers were used to implement allocation. Personnel at each study site used a central interactive voice response system to determine which container should be given to which participant. All study personnel, including investigators, site staff, participants, and sponsor staff remained blinded to treatment allocation throughout the study. Unblinding took place after data collection was complete. After being randomized, participants had up to two months to treat a qualifying migraine (as defined herein). Once the targeted number of treated participants with evaluable data was reached, study enrollment was stopped, as specified in the protocol at the sponsor's discretion. Participants who were enrolled but had not treated a migraine were asked to return their study medication and were discontinued from the study

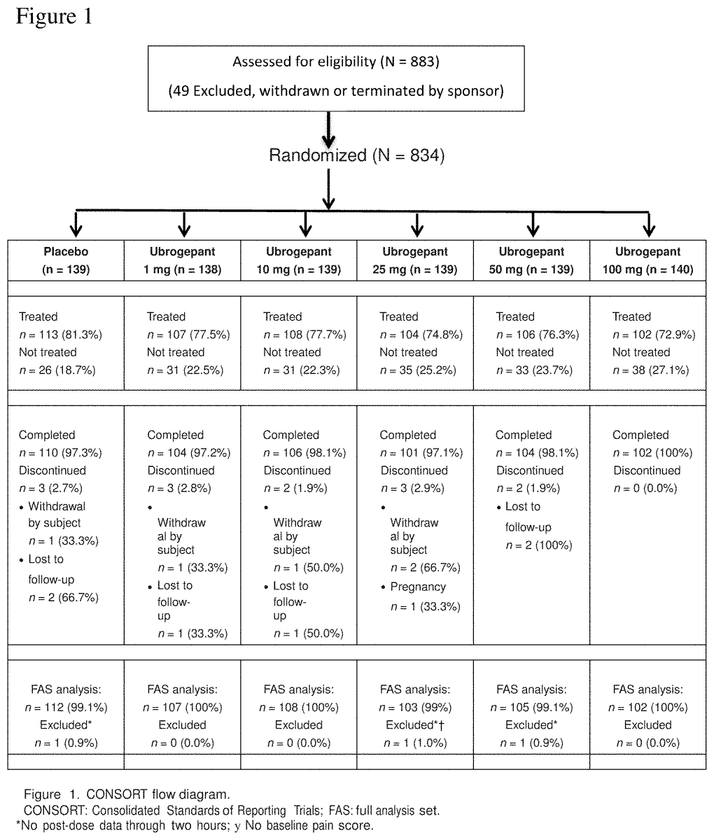

With reference to FIG. 1, eligible participants were allocated in a 1:1 ratio to one of the following treatment groups: 1, 10, 25, 50, and 100 mg of ubrogepant, or placebo. Study medication was provided to participants in a single bottle, with a single dose per bottle. Participants were instructed to take the dose when they experienced a qualifying migraine defined by the following conditions: migraine headache was moderate or severe (grade 2 or 3 on pain scale); migraine headache started less than four hours earlier; the migraine headache was a new headache that had not been previously treated and was not a recurrence of a previous migraine head ache (at least 48 hours had elapsed since the complete termination of their last attack (pain and all associated symptoms); no other migraine headache or headache had occurred in the previous 48 hours; the migraine headache was not already resolving on its own; and no prohibited medication had been taken.

The onset of migraine and the results from administering the study medication were self-reported by participants using a paper diary. Headache severity was measured on the following pain scale: 0 none; 1 mild; 2 moderate; 3 severe. Participants assessed headache at baseline and then every 30 minutes up to two hours, then hourly up to four hours and at 6,8,24, and 48 hours. The primary efficacy endpoints were pain freedom at two hours post-dose (reduction in headache severity from grade 2 or 3 at baseline to grade 0) and headache response at two hours post-dose (reduction in headache severity from grade 2 or 3 at baseline to grade 1 or 0). The secondary efficacy endpoints were: (1) absence of phonophobia, photophobia, nausea at two hours post-dose; (2) sustained pain freedom from 2-24 hours and from 2-48 hours post-dose (defined as pain freedom at two hours post-dose, with no administration of any rescue medication and no occurrence of a mild, moderate, or severe headache during the respective period after dosing with the study medication); (3) sustained pain relief from 2-24 hours and from 2-48 hours post-dose (defined as headache response at two hours post-dose, with no administration of any rescue medication and with no occurrence of a moderate or severe headache during the respective period after dosing with the study medication); (4) total migraine freedom at two hours post-dose (defined as pain freedom with no photophobia, phonophobia, nausea, or vomiting at two hours post-dose); (5) total migraine freedom from 2-24 hours and from 2-48 hours post-dose (defined as sustained pain freedom with no photophobia, phonophobia, nausea, or vomiting during the specified time period).

Participants were also monitored for safety endpoints, including standard safety assessment (medical history and physical examination; vital signs; electrocardiogram; laboratory tests including hematology, blood chemistry, and urinalysis; and female participants of childbearing potential were administered pregnancy tests) and adverse events, including any post-screening elevation in participants of aspartate amino-transferase (AST) and/or ALT, defined as: >3×upper-limit-of-normal (>3×ULN).

For the analysis of efficacy data, the Full-Analysis-Set population served as the primary population. For each two-hour endpoint (pain freedom, headache response, absence of symptoms), the minimum requirement for inclusion in the FAS population was that participants were administered study treatment, had a baseline headache severity measurement, and had at least one post-dose efficacy measurement prior to, or including, the two-hour time point. The All-Subjects-as-Treated (ASaT) population was used for safety analyses.

There were three primary efficacy hypotheses: (1) There is a positive response trend across ubrogepant doses, as measured by the proportion of participants with two-hour pain freedom; (2) at least one ubrogepant dose is superior to placebo in the treatment of acute migraine, as measured by the proportion of participants with pain freedom at two hours; and (3) at least one ubrogepant dose is superior to placebo in the treatment of acute migraine, as measured by the proportion of participants with headache response at two hours.

The dose-response trend test on the response proportions of two-hour pain freedom was tested first and served as the gatekeeper for subsequent pair-wise tests between ubrogepant doses and placebo. The closed testing procedure was then applied to each of the two co-primary hypothesis tests at significance level a 0.05 for the comparisons of the highest dose of ubrogepant to placebo in the following order: two-hour pain freedom and two-hour headache response. The overall treatment trend test was based on a logistic regression model with continuous covariates for treatment group and age and categorical covariates for oral triptan use stratum (high responder, low responder, or naive; as mentioned above under Study Design) and baseline headache severity (moderate or severe). The comparison of ubrogepant doses vs placebo were conducted using the appropriate pairwise contrasts within the logistic regression model. No trend tests were performed for the secondary endpoints, but the analysis methods were otherwise similar to those for the primary endpoints.

The planned sample size of 810 randomized participants (135 per group) had at least 84% power to demonstrate a positive dose response of two-hour pain freedom across all ubrogepant doses using a two-sided, 5% alpha-level test across a number of scenarios evaluated in a comprehensive simulation study. Power calculations were based on the assumption of a non-evaluable rate of no more than 25% and were adjusted for the planned interim analysis for futility evaluation. One interim analysis for futility was performed and reviewed by a standing internal data monitoring committee. All statistical analyses were performed using SAS version 9.3 (SAS Institute, Cary, N.C., USA).

### Presentation and Discussion of Trial Results

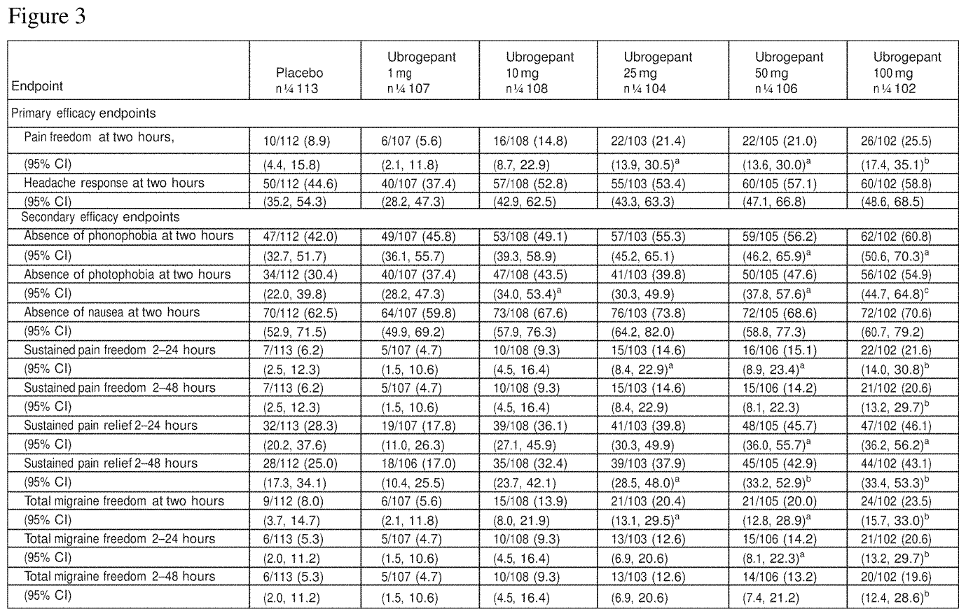

With reference to FIG. 1, a total of 834 participants were selected and randomized to the following groups: ubrogepant: n=138 to 1 mg; n=139 to 10 mg; n=139 to 25 mg; n=139 to 50 mg; and n=140 to 100 mg, and Placebo: n=139. The trial was conducted at 55 study centers in the United States in last half of calendar year 2012 (for more details, see T. Voss et al., Cephalalgia. 2016 Jun 6. pii: 0333102416653233, Epub ahead of print, Jun. 6, 2016), which publication is incorporated herein in its entirety by reference. With further reference to FIG. 1, of those randomized, 76.7% were administered study treatment, of those, 98.0% completed the study. FIG. 2 reports the baseline symptoms reported by participants prior to dosing at the onset of migraine, including the report of severity of pain experienced prior to dosing. FIG. 3 presents a summary of reports by participants of the level of severity of various symptoms at various times post-dosing, including “Pain Freedom” at two hours and “Headache Response” two-hours.

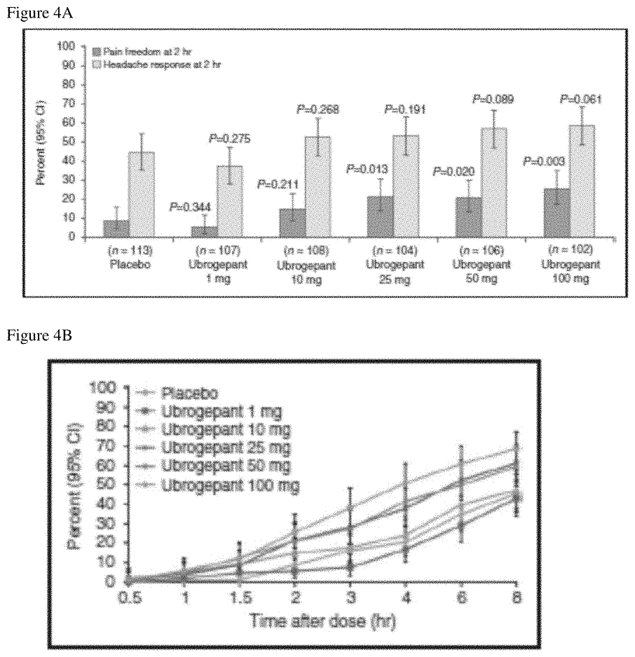

These data indicate that two of the three primary hypotheses were supported. There was a positive response trend across ubrogepant doses as measured by the proportion of participants who achieved two-hour pain freedom (p<0.001 for trend test). Pairwise comparisons were then performed for the highest dose vs placebo. Ubrogepant 100 mg demonstrated statistically significant superiority to placebo for two-hour pain freedom, supporting the second primary hypothesis. For the third primary hypothesis, two-hour headache response did not significantly differ between ubrogepant 100 mg and placebo. In accordance with the specified multiplicity strategy, this non-significant result precluded all additional hypothesis tests from having the possibility of being formally statistically significant, however, nominal p values (i.e. unadjusted p values) are used to further characterize these results. These results are presented in FIG. 4. As can be seen, this analysis indicates that 100 mg ubrogepant demonstrated a statistically significantly higher success rate than placebo and 50 mg and 25 mg ubrogepant demonstrated a nominally significantly higher two-hour pain-free rate than placebo. Additionally, 100 mg ubrogepant alone demonstrated a higher response rate compared to placebo for the co-primary two-hour headache response endpoint but was not deemed statistically significant.

Examining the secondary endpoint data, 100 mg ubrogepant demonstrated nominally significant improvements vs placebo on all secondary endpoints except absence of nausea at two hours (see FIG. 3). Nominally significant differences from placebo were also seen on the majority of secondary end-points for ubrogepant 50 mg (absence of photophobia at two hours, absence of phonophobia at two hours, sustained pain freedom 2-24 hours, sustained pain relief 2-24 hours, sustained pain relief 2-48 hours, total migraine freedom at two hours, total migraine freedom 2-24 hours) and on some secondary endpoints for 25 mg ubrogepant (sustained pain freedom 2-24 hours, sustained pain relief 2-48 hours, total migraine freedom at two hours). The proportions of participants meeting the pain freedom endpoint over the first eight hours after dosing (the time period over which hourly or half-hourly assessments were made) are shown in FIG. 3 and illustrate the time course of ubrogepant effects. Adverse effects (AEs) observed within 48 hours post-dose were similar for ubrogepant groups and placebo. No AEs showed clear dose dependence and the incidence of AEs within the 14 days post-dose was generally similar to the incidence within 48 hours. There were no observed post-treatment elevations of ALT >3×ULN and no other abnormal laboratory values of clinical relevance.

The foregoing indicates that when examining all efficacy data, these data were consistent positive treatment effects observed for the 25-mg, 50-mg, and 100-mg doses of ubrogepant across most endpoints, suggesting that all three of these doses may be efficacious.

Incorporated by reference herein also is the data from a pharmacokinetic study of ubrogepant for the acute treatment of migraine published on-line at (ClinicalTrials.gov: NCT01657370).

No significant CV events were observed and the AE profile was similar across subgroups categorized on the basis of low, moderate, or high coronary heart disease risk. However, participants with actual CV disease were not included in the study and conclusions regarding the safety of ubrogepant in such populations cannot be drawn from this single-attack study. There were no liver enzyme increases clearly attributable to administration of ubrogepant in the present single-dose study.

While the foregoing specification teaches the principles of the present invention, with examples provided for the purpose of illustration, the practice of the invention encompasses all of the usual variations, adaptations and/or modifications that come within the scope of the following claims.

What is claimed is:

1. A method for acute treatment of migraine, the method comprising oral administration to a patient suffering from a migraine attack 50 mg of ubrogepant in a solid oral dosage form,
wherein the method does not comprise further administration of ubrogepant to the patient within 2 hours from said oral administration of 50 mg of ubrogepant, and
wherein the patient achieves headache pain freedom at 2 hours post said administration of 50 mg ubrogepant.

2. The method of claim 1, wherein said solid oral dosage form comprises spray-dried ubrogepant.

3. The method of claim 1, wherein said solid oral dosage form comprises dispersible granules that comprise ubrogepant.

4. The method of claim 2, wherein said solid oral dosage form further comprises silicon dioxide.

5. The method of claim 2, wherein said solid oral dosage form further comprises microcrystalline cellulose.

6. The method of claim 2, wherein said solid oral dosage form further comprises croscarmellose sodium.

7. The method of claim 2, wherein said solid oral dosage form further comprises magnesium stearate.

8. The method of claim 1, wherein the patient had severe headache pain before the treatment.

9. The method of claim 8, wherein said solid oral dosage form comprises spray-dried ubrogepant.

10. The method of claim 8, wherein said solid oral dosage form comprises dispersible granules that comprise ubrogepant.

11. The method of claim 1, wherein the patient had moderate headache pain before the treatment.

12. The method of claim 11, wherein said solid oral dosage form comprises spray-dried ubrogepant.

13. The method of claim 11, wherein said solid oral dosage form comprises dispersible granules that comprise ubrogepant.

14. A method for acute treatment of migraine, the method comprising administering to a patient suffering an acute migraine 100 mg of ubrogepant in a solid oral dosage form,
wherein the method does not comprise further administration of ubrogepant to the patient within 2 hours from administering said 50 mg of ubrogepant, and
wherein the patient achieves pain freedom at 2 hours post administration of said 50 mg ubrogepant.

15. The method of claim 14, wherein said solid oral dosage form comprises spray-dried ubrogepant.

16. The method of claim 14, wherein said solid oral dosage form comprises dispersible granules that comprise ubrogepant.

17. The method of claim 15, wherein said solid oral dosage form further comprises silicon dioxide.

18. The method of claim 15, wherein said solid oral dosage form further comprises microcrystalline cellulose.

19. The method of claim 15, wherein said solid oral dosage form further comprises croscarmellose sodium.

20. The method of claim 14, wherein the patient had severe headache before the treatment.

21. The method of claim 20, wherein said solid oral dosage form comprises spray-dried ubrogepant.

22. The method of claim 20, wherein said solid oral dosage form comprises dispersible granules that comprise ubrogepant.

23. The method of claim 14, wherein the patient had moderate headache before the treatment.

24. The method of claim 23, wherein said solid oral dosage form comprises spray-dried ubrogepant.

25. The method of claim 23, wherein said solid oral dosage form comprises dispersible granules that comprise ubrogepant.
